(19) United States
(12) Patent Application Publication
(10) Pub. No.: US 2003/0171950 A1
(43) Pub. Date: Sep. 11, 2003

(54) Inventory management method and system for prescribed goods and services

(76) Inventors: Conor Kilgannon, Springfield, NJ (US); Thomas Burke, Oldwick, NJ (US)

Correspondence Address: Stephen G. Matzuk, P.O. Box 767, Boston, MA, 02102, US

(21) Appl. No.: 10/090,657

(22) Filed: Mar. 5, 2002

(51) Int. Cl. G06F 17/60

(52) U.S. Cl. 705/2

(57) ABSTRACT

A comprehensive program method and system particularly adapted for the segments of the healthcare industry dealing with Durable Medical Equipment items and related out-patient services, such as found in the home health care segment, wherein the prescribed patient goods and services, 3rd party providers, and insurance companies are efficiently integrated according to various functions, e.g. pre-certification, purchasing, leasing, inventory management, billing coding of goods/services, fraud detection and prevention, and final delivery to the patient or consumer. According to one feature of the present invention, the advantages of ownership of the Durable Medical Equipment by the prescribing or insuring groups such as Health Maintenance Organizations, are provided without any of the maintenance or other headaches typically associated with ownership by bulk ordering and outsourcing the warehousing and shipping to facilities which may be shared by other prescribing or insuring groups, while avoiding the current system of repeatedly renting the DME item without receiving title.

### FIELD OF THE INVENTION

The present invention relates to inventory control systems, in particular, to inventory control of prescribed goods and services.

### BACKGROUND OF THE INVENTION

The normal free-market flow and associations between consumers and suppliers are interrupted by the interposition of third-party insurers, especially in the healthcare industries wherein the would-be consumers are patients, and their prospective purchases of goods and services are influenced or mandated by still other third parties (e.g. physicians) who may prescribe services or goods. The typical operations of the insurers, such as Health Maintenance Organizations (HMO), typically localize the demand for goods or services to fragmented markets in the immediate locale of the patient, inhibiting any significant market aggregation of the patient demands for goods or services.

While less noticeable for small goods or highly individualized services, such isolated and fragmented markets permit if not encourage elevated insurer costs. The Durable Medical Equipment, such as home hospital beds and other equipment, are generally rented to the patient for a duration of need. Typically, the insurer must endure unnecessarily elevated costs and often the consumer experiences either the unavailability or underavailability of the needed goods and services in the durable medical and home health care or medical equipment (HME) segment of the health care market.

Moreover, there is a lack of effective administration oversight. The patient is often left to the mercy of the local goods or services supplier, creating an opportunity to exploit the patient and introduce fraud, such as shipping the patient a lesser good even though contracted rates are in place. No information is provided to the insurer to more efficiently operate or serve the patient in the future. Thus, the patient costs are not subject to appropriate market minimizing due to an ineffective contacting strategy for the provision of DME and HME to the HCP member.

### SUMMARY OF THE INVENTION

The system and method according to the present invention provides to the insurer, a common warehouse of Durable Medical Equipment and coordinated logistical nation-wide distribution of such goods, or coordination of third-party services for the patient, thus permitting market aggregation in economies having third-party prescriber and insurers components associated with, limiting or in some cases driving consumer demands for goods or services to provided lower cost goods and services, while increasing the availability thereof, while providing the benefit of ownership to the insurer and ultimately to the patient.

Moreover, the system and method according to the present invention further reduces losses due to fraud and mismanagement of resources. Furthermore, the system and method according to the present invention permits the aggregation of market information and data to provide better patient logistical support and anticipation of present and future patient needs.

The present invention also allows smooth transition of present federally administered provider systems to an efficient privatized structure, which can be easily legislatively monitored, while maximizing free-market leverage to minimized patient costs.

### BRIEF DESCRIPTION OF THE DRAWING

These and further features of the present invention will be better understood by reading the following Detailed Description together with the Drawing, wherein

FIG. 1 is a block diagram of one embodiment of the present invention; and

FIG. 2 is a flow chart of one embodiment of the present invention.

### DETAILED DESCRIPTION OF THE PRESENT INVENTION

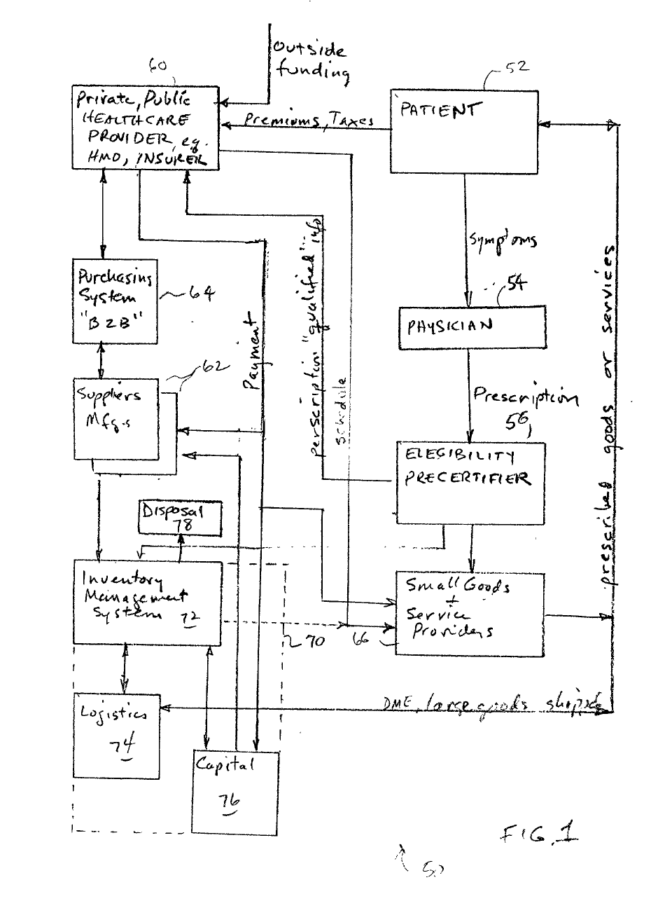

The preferred embodiment 50 of the present invention is shown in FIG. 1, wherein a patient 52 is enrolled in a health care provider system 60, e.g. a private or public Health Care Provider (HCP) insurer, and pays premiums and/or taxes to enable the HCP to provide managed resources for the prescribed medical needs, e.g. goods and services, for a plurality of patients 52. An individual patient 52 presents to a physician, clinic, etc. 54 with symptoms or complaints, for which goods and/or services are prescribed. Typically, the goods include small and/or disposable goods, large and/or Durable Medica Equipment (DME), and specific 3rd-party services, e.g. physical therapists, orthotic fitters, etc. The prescription for goods and/or services is evaluated by an eligibility prescriber system 56, e.g. Medunite (TM) or the existing pre-certification department which evaluates claimed prescribed goods/services under HCP patient policies. If the prescribed goods and/or services are eligible under the patient's HCP policy, the HCP is notified and the corresponding source of goods or services are notified.

For small and/or disposable goods, nationally contracted uniform supply outlet(s), e.g. national drug store(s), representatives and suppliers 66 are notified of the patient need, for which the patient is supplied and the costs therefore being reimbursed by the HCP. Moreover, specific services, e.g. therapist and medical appliance fitters from one or more available agencies 66 are notified and scheduled for rendering services to the patient, and paid for by the HCP 60.

Acquisition of larger or Durable Medical Equipment (DME) is provided by a purchasing system 64 and inventoried (when received from the suppliers and manufacturers 62) by a system 70 distinct from the HCP 60, but operated for the use of the HCP 60 by its managing system 72, internal logistics 74, e.g. DME distribution, storage and shipping, and capital operation 76 which provides financing of purchased resources, etc. by capital resources and resource management operation. Moreover, the inventory management system 72, logistics and distribution 74, and capital resources 76 are integrated into a unified operation 70 (also available to others for other purposes), such as exemplified by United Parcel Services which includes UPS logistics and UPS capital groups, which optionally provides capital 76 and storage logistics 74 as well as inventory management 72 in addition to direct-to-patient shipping. The approved DME is provided to the patient from a selected distribution center, part of the logistics 74, for the duration of patient need, after which it is returned to a selected logistics reception center, refurbished and redistributed for the duration of the economic life of the DME. After the lifetime of the DME, it is disposed of by waste recycling, charitable or other use 78.

The DME is acquired by the system 50 according to one embodiment of the present invention by reviewing patient needs or leasing such items in order to secure the benefits (e.g. tax, lower costs, depreciation etc.) of DME ownership with an acquisition system 64 which engages all suppliers 62 in competitive bidding processes between the supplier(s) and the DME, i.e. as a business-to-business (“B-2-B”) system and process, such as the “Ariba” system. The purchased DME goods are received by the inventory management system 72 and stored for distribution at one of several distribution centers located geographically to serve the patient needs as determined by the HCP 60 according to prior and present prescription requests. Systems for matching of prescription to item billed, for billing term inaccuracies, overbilling, etc., and (re)checking member eligibility, and/or for (redundant) checking for an oversupply of item to member is also included in the unified system 70, either solely within the inventory management system 72 or distributed to other constituent systems.

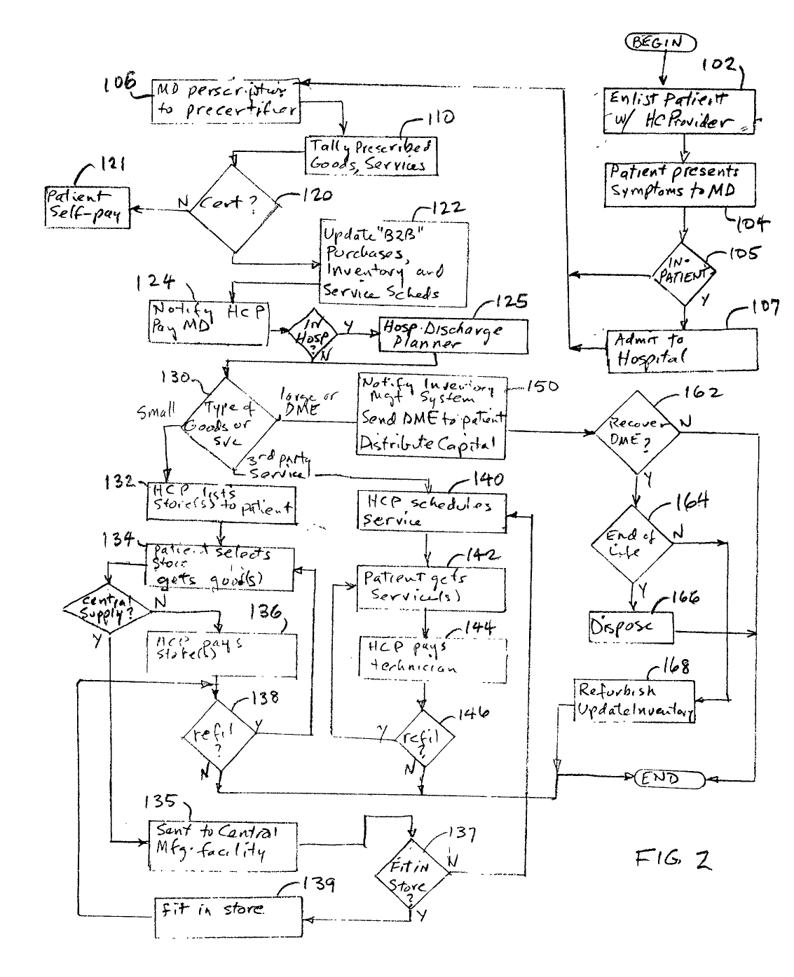

The management of the HCP resources and prescribed patient goods and services is illustrated by the process 100 of FIG. 2. The patient is enlisted 102 in a private or public health care system or health care provider (HCP) to which premiums are paid, or taxes are paid for public HCPs, or outside funding is received. The HCP income is managed for future client patient needs. When the client patient presents to an MD with symptoms, 104, and the patient is not admitted to a hospital as an “in-patient” step 105, the patient is given a prescription for goods and/or therapeutic services, 106. Before the resources are released by the HCP or the prescription filled, the prescription is pre-certified, 120, if necessary (e.g if >$200) by a system which includes outsourced third-party evaluation services, e.g. Medunite (TM), and a programmed evaluation system resident in computation facilities available or part of the HCP. If the patient is admitted to the hospital as an “in-patient”, the hospital forwards at step 107, the prescription of goods or services to the pre-certifier at step 106. If the prescription fails to pre-certify, the patient is advised and may elect to self-pay 122. If the prescription is certified, the physician is paid (if participating), 124, and the prescription is forwarded for filling.

Simultaneously, the patient prescription needs are monitored, tallied 110. If national or geographically significant regional demands for particular prescription goods or services exist, as determined by an evaluation system also optionally including business forecasting software, e.g. as provided by UPS Logistics Group, the HCP may elect to schedule, purchase, manufacture, etc. such goods for its own ownership and client patient consumption. Thus according to one feature according to the present invention, such goods (or services) are purchased (or contracted) by the HCP directly from other supplier businesses in a business-to-business (“B-2-B”) arrangement. Such B-2-B arrangements are made via competitive bidding or other cost or performance optimizing processes, such as the “Ariba” system or other similar systems, e.g. Commerce One, Oracle. The purchased goods are received and distributed for good regional patient proximity and inventories of available goods and services updated, 122.

Small and/or disposable goods are provided by a HCP listed store or stores, typically nationally contracted uniform suppliers, at step 132 which store(s) may also contract out for the specific prescription patient need as authorized by the HCP. When more than one store is approved by the HCP, the patient selects the store and receives the prescription, 134, and the store is paid by the HCP at step 136. If the prescription is to be refilled as determined at step 138, the patient selected store refills the prescription; otherwise, the process is complete for that particular client patient and prescription item. For patients admitted to the hospital in step 105, once purchases, inventory and service schedules are updated, step 122, and HCP is notified and MD (and hospital, etc. are paid, step 124, a hospital discharge planner (or equivalent) coordinates the subsequent “out-patient” prescription goods and services arrangements, steps 130 et seq. beginning at the hospital discharge date, and according to information provided at step 125.

Prescription services are similarly provided according to the system and method of the present invention, wherein patient services, such as therapists and orthotic fitters, are identified and scheduled at step 140, and the services are rendered or delivered to the patient at step 142. After step 140, the patient measurements for orthotic devices, etc., are taken at to the selected store at step 134 sent to central manufacturing facility at step 135, and shipped back to the selected store for fitting at step 139, or at a scheduled MD's (or service technician's) office for fitting at step 140. The service technician is paid, 144, and if the service is to be repeated as determined by step 146, the process repeats and the prescribed service is again rendered at step 142; otherwise, the process is complete for that particular client patient and prescription service(s).

A further feature according to the present invention is the ownership acquisition of large or Durable Medical Equipment (DME) by the HCP, which has been purchased (and then leased using UPS capital) as described in step 122, above to avoid large capital outlays. The DME is distributed and shipped to the client according to the HCP-certified prescription at step 150. If, after the patient ends the use of the DME, step 162, there is any remaining economic life in the DME, step 164, the DME is returned, refurbished and the inventory is updated at step 168. If the recovered DME has reached the end of its life, it is disposed of, step 166. The process is complete for that particular client patient and prescription large and DME items.

These and further alternate embodiments, and modifications and substitutions according to one of ordinary skill in the art are considered to be within the scope of the present invention, which is not to be limited except as claimed.

What is claimed is:

1. An inventory management system for patient goods and services as prescribed by a prescribing party, comprising:
a funding resource center having a resource control system therein, for receiving patient funds, and for allocating at least one of patient funds, purchased third-party goods and purchased third-party services according to said resource control system;
an eligibility processing center for receiving patient claim and patient evaluator information, and providing patient qualification determination information to said resource center; and
an aggregate resource center having a contracting system for optimizing the delivery of third party goods and services according to qualified prescribed patient needs and logistics, said aggregate resource center also including a goods warehouse and distribution system for conveying said qualified prescribed patient needs and services.

2. The system of claim 1, wherein said funding resource center comprises one of a private healthcare provider, a public healthcare provider and an insurance company.

3. The system of claim 1, wherein said funding resource center includes a business-to-business purchasing system for purchasing goods from at least one of a manufacturer and a supplier.

4. The system of claim 3, wherein said purchasing system includes a system for shipping goods acquired by said funding resource center from said manufacture and said supplier to said aggregate resource center.

5. The system of claim 1, wherein said aggregate resource center includes a capital funding source.

6. The system of claim 1, wherein said aggregate resource center comprises a third party.

7. The system of claim 1, further including small goods and service providers.

8. The system of claim 7, wherein one of said funding resource center and said aggregate resource center includes a service provider scheduler.

9. The system of claim 1, further including a disposal system for disposing of goods which have exceeded their economic lifetime.

10. The system of claim 1, wherein said eligibility processing center comprises a third party eligibility processing center.

11. A method of managing inventory and distribution of patient goods and services as prescribed by a prescribing party, comprising the steps of:
controlling patient resources including the steps of receiving money for said patient and distributing said money upon receipt of a prescription request and eligibility pre-certification;
pre-certifying patient prescriptions for disbursement of patient resources by comparing said patient prescription to a predetermined pre-certification requirement; and
distributing to said patient at least one of goods and services in response to money distributed by said step of controlling patient resources.

12. The method of claim 11, further including the step of purchasing goods for distribution to said patient.

13. The method of claim 12, wherein said step of purchasing goods comprises the step of business-to-business purchasing goods.

14. The method of claim 12, further comprising the steps of:
receiving goods back from said patient, and
updating system inventory.

15. The method of claim 14, further including the step of redistributing the goods to a subsequent patient.

16. The method of claim 11, further including the step of scheduling the delivery of said goods and service according to said step of controlling patient resources.

17. The method of claim 16, further including the step of coordinating the delivery of goods and services with the discharge of said patient from a hospital.

18. The method of claim 16, wherein said services comprises one of third party therapy and third party technical services.

19. The method of claim 16, wherein the step of scheduling delivery includes the step of scheduling payment to providers of said goods and services.

20. The method of claim 11, wherein said step of distributing further includes the steps of
selecting a plurality of third-party source of said goods and services,
patient selecting of one of said plurality of third party sources, and
distributing patient money thereto for rendering prescribed goods and services to said patient.

21. The method of claim 11, further including the steps of:
determining the need of goods from prior patient prescriptions, and
centrally manufacturing said goods in a quantity according to said prior patient need.

22. The method of claim 11, further including the steps of:
anticipating patient needs according to prior patient needs, and
geographically distributing said goods according to the anticipated patient needs.
